(19) United States
(12) Patent Application Publication
(10) Pub. No.: US 2026/0183196 A1
(43) Pub. Date: Jul. 2, 2026

(54) SYSTEM FOR TRACKING AND ANALYZING FUNCTIONAL FEEDING

(71) Applicant: Solica Connect, Inc., Melrose, MA (US)

(72) Inventors: Nancy Alissa McFall, Watertown, MA (US); Soniya Patel, Watertown, MA (US); Cassie Moreira, Melrose, MA (US)

(21) Appl. No.: 19/429,543

(22) Filed: Dec. 22, 2025

Related U.S. Application Data
(60) Provisional application No. 63/739,020, filed on Dec. 26, 2024.

(51) Int. Cl. A61J 9/00; A61B 5/00; A61B 90/00

(52) U.S. Cl. CPC A61J 9/00; A61B 5/7203; A61B 5/7275; A61B 5/742; A61B 90/06; A61B 2090/064; A61B 2503/04; A61J 2200/70

(57) ABSTRACT

A system for tracking feeding activity of a baby includes a housing configured to be positioned over an object for feeding the baby. A plurality of sensors are disposed on or within the housing, the sensors being configured to detect pressure exerted by one or more oral structures of the baby during feeding. A data collection device is operatively coupled to the plurality of sensors, and configured to receive sensor signals and process the signals to determine one or more feeding metrics. The data collection device outputs the one or more feeding metrics for display to a user, wherein the feeding metrics include one or more of: latch quality, tongue movement, lip seal integrity, or suck-swallow-breathe coordination.

### CROSS REFERENCE TO RELATED APPLICATIONS

This application claims the benefit of U.S. Provisional Patent Application No. 63/739,020, filed Dec. 26, 2024, the contents of which are incorporated herein by reference in their entirety as if fully set forth.

### FIELD

Illustrative embodiments of the invention generally relate to nursing and, more particularly, various embodiments of the invention relate to using objective criteria relating to baby movement to manage and track feeding in nursing or bottle feeding.

### BACKGROUND

In the United States and around the world, feeding challenges are quite common among infants in the first six months of life, with a significant number experiencing difficulties with breastfeeding, bottle-feeding, or both.

Most U.S. mothers begin breastfeeding at birth. However, a significantly smaller number continue to breastfeed at six months. Exclusive breastfeeding rates drop considerably by this time due to factors such as low milk supply, latch issues, or the need to return to work. Additionally, many breastfeeding mothers experience challenges like nipple pain, breast engorgement, or infections such as mastitis in the early months, which can impact their ability to maintain breastfeeding.

Feeding-related issues are a significant source of anxiety for parents, with many new parents reporting stress or uncertainty around feeding choices and frequency. Feeding problems can exacerbate postpartum depression and impact bonding between parent and infant. While breastfeeding support is increasing, there remains a gap in feeding support for lactation support and adaptive feeding tools, as only a minority of mothers report receiving adequate guidance on feeding options and techniques. Flow rates that are too fast or too slow contribute to feeding discomfort and potential digestive issues. Compounding problems, many babies show nipple confusion, making it difficult to switch between feeding methods.

Addressing these challenges requires both effective interventions and innovations in feeding devices to better support all types of feeding needs skills to reduce feeding-related stress. The current way to measure and test a baby's functional feeding is through subjective observation with an available professional, such as a trained professional.

### SUMMARY OF VARIOUS EMBODIMENTS

In accordance with an embodiment, a system for tracking feeding activity of a baby includes a housing configured to be positioned over an object for feeding the baby. A plurality of sensors are disposed on or within the housing, the sensors being configured to detect pressure exerted by one or more oral structures of the baby during feeding. A data collection device is operatively coupled to the plurality of sensors, and configured to receive sensor signals and process the signals to determine one or more feeding metrics. The data collection device outputs the one or more feeding metrics for display to a user, wherein the feeding metrics include one or more of: latch quality, tongue movement, lip seal integrity, or suck-swallow-breathe coordination.

In some embodiments, the housing is configured to be placed over a human breast.

In some embodiments, the housing is configured to be incorporated into a nipple of a baby feeding bottle.

In some embodiments, the housing includes a first portion proximate to an opening for fluid communication, a second portion radially outward from the first portion, and a third portion distal from the opening, the plurality of sensors being distributed across the first and second portions.

In some embodiments, the plurality of sensors includes a first sensor positioned to detect pressure from a posterior tongue region, a second sensor positioned to detect pressure from an anterior tongue region, a third sensor positioned to detect pressure from a lower lip region, and a fourth sensor positioned to detect pressure from an upper lip region.

In some embodiments, the sensors comprise at least one of a fluid-filled sensor configured to convert pressure-induced deformation into an electrical signal, or a microelectromechanical system (MEMS) sensor embedded within a flexible housing material.

In some embodiments, the system includes a pressure measuring sensor system that measures change in muscle pressure, wherein the pressure measuring sensor system measures one or more of the following: resistance, capacitance, or inductance changes in the baby's orofacial muscle mechanics.

In some embodiments, the system includes one or more holes adapted to facilitate biochemical feedback.

In some embodiments, the data collection device includes signal conditioning circuitry configured to filter noise, normalize sensor outputs, and compute pressure ratios between tongue and lip regions.

In some embodiments, the data collection device is configured to generate a composite feeding score based on empirical data, clinical benchmarks, or machine learning algorithms.

In some embodiments, the system further includes a display configured to present real-time feedback including graphical plots of pressure profiles and rhythmic feeding patterns.

In some embodiments, the system further includes a tongue movement isolation module configured to prevent interference between tongue and lip pressure signals during feeding analysis.

In accordance with an embodiment, a method for assessing feeding activity of a baby includes positioning a housing over a feeding interface configured to contact one or more oral structures of the baby. Via a plurality of sensors disposed on or within the housing, pressures exerted by the baby during feeding are detected. Sensor signals are transmitted from the plurality of sensors to a data collection module. The sensor signals are processed to generate one or more feeding metrics based on detected pressure patterns, and output for presentation to a user, the feeding metrics comprising at least one of latch quality, tongue movement, lip seal integrity, or suck-swallow-breathe coordination.

In some embodiments, positioning the housing includes positioning the housing over a human breast.

In some embodiments, positioning the housing includes integrating the housing into a nipple of a baby feeding bottle.

In some embodiments, detecting pressures includes detecting pressures across a proximal region near a fluid opening and an intermediate region radially outward from the proximal region.

In some embodiments, detecting pressures includes detecting pressure applied by a posterior region of the baby's tongue, detecting pressure applied by an anterior region of the baby's tongue, detecting pressure applied by the baby's lower lip, and detecting pressure applied by the baby's upper lip.

In some embodiments, detecting pressures includes receiving signals from at least one of a fluid-filled pressure sensor configured to convert deformation into an electrical response, or a MEMS-based pressure sensor embedded in a flexible portion of the housing.

In some embodiments, the method further includes conditioning the sensor signals by filtering noise, normalizing amplitudes, and calculating pressure ratios between tongue-associated and lip-associated sensors.

In some embodiments, processing the sensor signals includes generating a composite feeding score using empirical training sets, clinical reference values, or a machine-learning model.

In some embodiments, the method further includes displaying real-time graphical representations of pressure profiles, rhythmic feeding cycles, or time-based feeding trends.

Illustrative embodiments of the invention are implemented as a computer program product having a computer usable medium with computer readable program code thereon. The computer readable code may be read and utilized by a computer system in accordance with conventional processes.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 shows an example system for tracking nursing in accordance with illustrative embodiments.

FIG. 2 shows an example schematic diagram of the system of FIG. 1 located on a breast during nursing of a baby in accordance with illustrative embodiments.

FIG. 3 shows an example schematic diagram of the system of FIG. 1 located on a bottle during nursing of a baby in accordance with illustrative embodiments.

FIG. 4 shows an example process of tracking feeding in accordance with illustrative embodiments.

FIG. 5 shows an example schematic block diagram of the system of FIG. 1 in accordance with illustrative embodiments.

FIG. 6 shows an example user interface with data available to parents/professionals to review data collected in accordance with illustrative embodiments.

FIG. 7 shows an exemplary waveform with annotations of a swallowing and breathing pattern for a baby while breastfeeding in accordance with illustrative embodiments.

FIG. 8 shows an exemplary plot of ideal ratio of feeding phases over development time in accordance with illustrative embodiments.

FIG. 9 shows an exemplary plot of expected pressure of lip and tongue during one sequence of feeding in accordance with illustrative embodiments.

FIG. 10 shows an example computing device in accordance with illustrative embodiments.

### DESCRIPTION OF ILLUSTRATIVE EMBODIMENTS

Illustrative embodiments gather objective dynamic information about a baby while feeding/nursing and, using that information, enable professionals to optimize and/or improve feedings when dysfunction is present, as well as to monitor function as the baby grows and develops. To that end, in illustrative embodiments, a system to track nursing operates as a feeding monitor to measure a baby's functional movement during breastfeeding to share what is objectively happening. The system/monitor measures one or more of muscle pressure, muscle movement, and pace patterns during a feeding session. Among other things, the system may analyze the function of the lips, anterior tongue and posterior tongue. These measurements enable parents and professionals to gather sufficient data relating to the baby's physical structure, taking much of the subjectivity out of treatment methods needed when dysfunction is present. Details of illustrative embodiments are discussed below.

As mentioned above, current techniques to measure and test a baby's functional feeding are through subjective observation. For example, current healthcare metrics focus on quantity (e.g., weight, BMI) rather than functional feeding mechanics. Further, there is a lack of differentiation between breastfed and bottle-fed infants in official guidelines (e.g., CDC), leading to misclassification and missed correlations with rising health issues.

When breastfeeding, a baby achieves a deep latch by taking in both the nipple and the areola, forming a secure seal. This process involves a combination of suckling, suction, and tongue movement. The baby's tongue cups and compresses the breast tissue to stimulate milk flow. Through rhythmic, wave-like motions, the baby compresses the milk ducts using their gums and tongue while creating a vacuum to extract milk. Proper coordination of sucking, swallowing, and breathing is essential, as the baby must pause to breathe to properly intake enough nutrition.

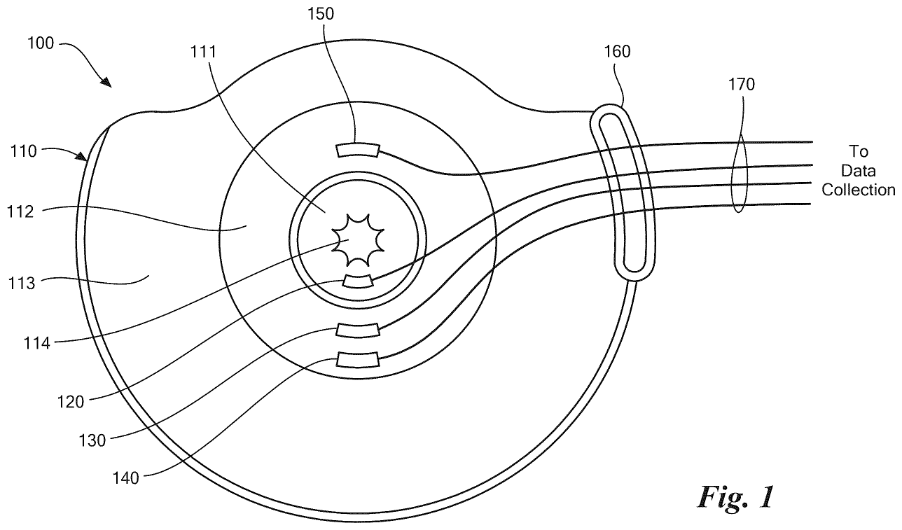

FIG. 1 illustrates an example system 100 for tracking nursing activity in accordance with various illustrative embodiments. The system 100 is designed to monitor feeding dynamics, such as pressure exerted during suckling, and transmit corresponding data for analysis. As shown, the system 100 includes a housing 110, which serves as the primary structural component. In certain embodiments, the housing 110 may be generally cylindrical, conical, or pyramidal in shape, and is configured to be positioned over a breast or integrated as a nipple component of a feeding bottle. This versatility allows the system to be used in both natural breastfeeding and bottle-feeding scenarios.

The housing 110 comprises multiple portions arranged along its longitudinal axis. A first portion 111 is located at the proximal end, closest to the baby's mouth during feeding. This first portion 111 includes an opening 114 that provides a fluid communication path for nourishment (e.g., breast milk or formula) from a source to the baby's mouth. Surrounding the opening 114, the first portion 111 may include a flexible material to mimic the tactile feel of a natural nipple, thereby promoting comfort and acceptance by the infant.

Adjacent to the first portion 111 is a second portion 112, which extends radially outward and provides structural support for sensor integration. A third portion 113 is positioned at the distal end of the housing 110, opposite the opening 114. In certain embodiments, the first, second, and third portions (111, 112, and 113) are generally circular and arranged concentrically about the opening 114. The third portion 113 may exhibit an asymmetric profile, such that its lower half has a greater outer radius compared to its upper half, thereby facilitating ergonomic placement against the breast or bottle surface.

The system 100 incorporates multiple sensors for detecting pressure variations during feeding. A first sensor 120 is disposed on or within the first portion 111, enabling measurement of pressure exerted near the nipple opening. A second sensor 130 and a third sensor 140 are positioned on lower sections of the second portion 112, with the third sensor 140 located farther from the opening 114 than the second sensor 130. In various embodiments, a sensor grid (or array) may be utilized where more or less sensors are included.

Additionally, a fourth sensor 150 is located on an upper section of the second portion 112, generally aligned with the second sensor 130 in terms of radial distance from the opening 114. This distributed sensor arrangement enables multi-point pressure mapping, which can be used to infer suckling strength, feeding patterns, and potential anomalies.

In various embodiments, the sensors 120, 130, 140, and 150 may include one or more of the following technologies:

Fluid-Filled Sensors: These sensors utilize a flexible, fluid-filled (e.g., air or silicone-based) chamber coupled to a transducer that converts pressure-induced deformation into an electrical signal. This design allows sensitive pressure detection while maintaining electronic components at a safe distance from the baby's mouth.

MEMS or Thin-Film Sensors: Microelectromechanical systems (MEMS) or piezoresistive thin-film sensors may be embedded within the housing material (e.g., silicone). Such sensors offer high precision and compact form factors, making them suitable for integration into feeding devices.

Each sensor may be operatively connected to a transmission wire 170, which passes through a grommet 160 to maintain a sealed interface and prevent fluid ingress. The transmission wire 170 carries electrical signals to a data collection device, which may include processing circuitry for signal conditioning, data storage, and wireless communication. In some embodiments, the data collection device is integrated within the housing 110; in other embodiments, it is external and connected via wired or wireless interfaces. The collected data may be analyzed to determine feeding metrics such as pressure profiles, feeding duration, and suckling frequency, which can be used for health monitoring or behavioral studies.

In various embodiments, the housing 110 may be formed from a material with proven safety, durability, and widespread use in medical devices and baby product. For example, it may be formed, at least in part, from medical-grade silicone and BPA-free plastic. Medical-grade silicone is known for its biocompatibility, flexibility, and resistance to microbial growth, making it effective for direct skin contact and frequent use.

BPA-free plastic ensures structural integrity while maintaining a non-toxic, safe environment for users, aligning with health and safety standards. Together, these materials enhance user comfort, reliability, and compliance with medical and consumer safety regulations.

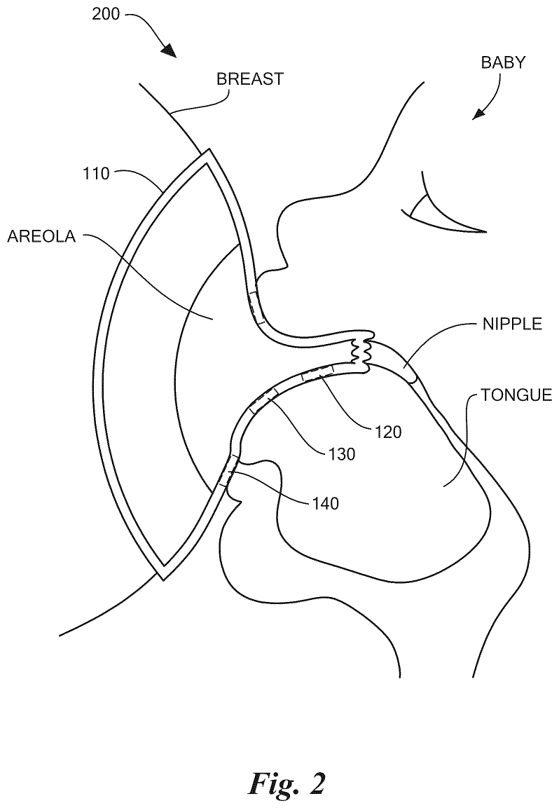

If tubing is required for a barometric sensor wire (e.g., 170), illustrative embodiments may use medical-grade thermoplastic elastomers (TPE) or flexible silicone tubing to ensure biocompatibility, durability, and precise pressure measurements. FIG. 2 illustrates an example schematic diagram 200 of the system 100 of FIG. 1 positioned on a breast during nursing of a baby, in accordance with various illustrative embodiments. As shown, the baby is actively nursing, with the nipple of the breast located inside the baby's oral cavity to fluidly communicate breast milk for nourishment. The system 100 is configured to monitor biomechanical interactions between the baby's mouth and the nipple during feeding.

In this embodiment, the housing 110 of the system 100 is positioned over the breast such that the first portion 111 aligns with the nipple tip and the opening 114 provides unobstructed milk flow. The sensors are strategically located to correspond to anatomical regions of the baby's mouth:

Sensor 120 is in contact with or proximate to the posterior tongue region, enabling detection of pressure variations associated with peristaltic tongue movements during suckling.

Sensor 130 is positioned to interact with the anterior tongue region, capturing pressure changes as the tongue elevates and compresses the nipple during latch formation.

Sensor 140 is aligned with the lower lip region, detecting compression forces and suction applied by the baby's lower lip.

Sensor 150 is aligned with the upper lip region, monitoring pressure exerted by the upper lip during feeding.

Each sensor measures pressure and/or suction forces at its respective location, allowing the system to generate a multi-point pressure profile. This profile can be analyzed to determine latch quality, feeding efficiency, and detect anomalies such as weak suckling or improper latch. In some embodiments, the sensors may also capture dynamic changes throughout the nursing cycle, including rhythmic patterns and peak pressure intervals.

The sensors communicate collected data to a data collection device via wired or wireless transmission. The data collection device may include processing circuitry configured to filter noise, compute pressure gradients, and store time-series data for subsequent analysis. In certain embodiments, the device may transmit data to a remote server or mobile application for real-time monitoring by caregivers or healthcare professionals.

In various embodiments, the system 100 may include one or more holes throughout to allow biochemical feedback where saliva passes through to communicate with the nipple.

In various embodiments, during breastfeeding, a baby's saliva enters the nipple and communicates with receptors in the mother's areola, signaling the baby's current needs. This biochemical feedback may aid the mother's body to adjust the milk in real time—changing immune factors, antibodies, and nutrient composition to better support the baby's health (e.g., responding to illness or growth needs). When this is blocked, it changes the functional and mechanical movements as baby and mother have to work harder to guess needs. Accordingly, a two-way biological conversation may be accomplished, in addition to feeding.

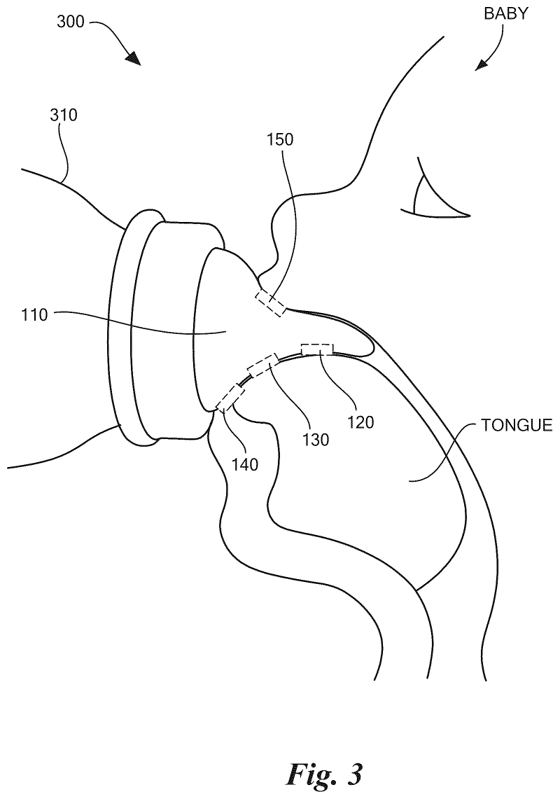

FIG. 3 illustrates an example schematic diagram 300 of the system 100 positioned on a bottle 310 during feeding of a baby, in accordance with various illustrative embodiments. Similar to FIG. 2, the baby is feeding with the nipple of the bottle located inside the baby's mouth to fluidly communicate nourishment. In one embodiment, the nipple of the bottle is integrally formed as the housing 110 of the system 100. In another embodiment, the housing 110 is adapted to fit over a conventional silicone nipple, enabling retrofitting of existing bottles.

As Shown, the Sensor Arrangement Mirrors that of FIG. 2:
Sensor 120 interfaces with the posterior tongue region.
Sensor 130 interfaces with the anterior tongue region.
Sensor 140 interfaces with the lower lip region.
Sensor 150 interfaces with the upper lip region.

This configuration ensures consistent data acquisition across both breastfeeding and bottle-feeding scenarios. Each sensor detects pressure and suction forces to assess latch quality and feeding dynamics. The system may further differentiate between natural and artificial feeding patterns by analyzing pressure distribution and frequency characteristics.

In various embodiments, the housing material may be food-grade silicone or thermoplastic elastomer to ensure safety and comfort. The sensors may be encapsulated within the housing to prevent fluid ingress and maintain hygiene. The data collection device may be integrated into the bottle cap or located externally, connected via a sealed grommet and transmission wire 170, or through wireless communication protocols such as Bluetooth® or NFC.

### Collected Data can be Used for Multiple Purposes, Including:

Clinical Monitoring: Detecting feeding difficulties, diagnosing oral-motor disorders, or tracking recovery post-surgery.
Behavioral Analysis: Studying suck-swallow-breathe coordination and feeding habits.
Parental Feedback: Providing real-time alerts or summaries via a mobile application.

In various embodiments described above and herein, after the baby latches, three pressure sensing regions around the opening gather information:
The outer region measuring lip pressure (lips to nipple measurement ˜3 cm);
the middle region measuring anterior tongue pressure (˜1.5 cm away from the nipple); and
the inner region measuring posterior tongue pressure (˜0.5 cm away from the nipple).

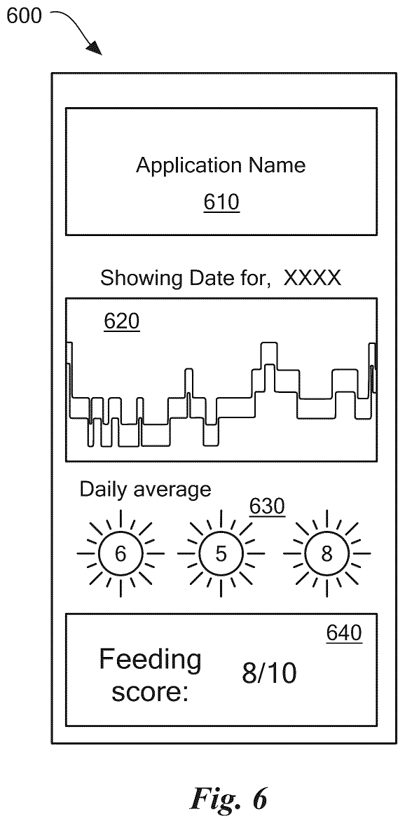

The three regions communicate with a processor to collect and process the information from the pressure sensors. Information can be displayed and entered via a simple display screen, the black box diagram can be referenced in FIG. 6 below. As the baby feeds, the system collects the pressure data of the three channels/sensors to provide data for analysis of the different muscle functions. This objective data then may be converted to a score for each function shown on the display screen. These scores can be determined in a number of manners, such as by a function or empirical data via a look up table. After receiving the score, parents and professionals can log the score and compare it to normative data, as well as data collected over time from the baby, to determine an individualized, objectively based normative functioning level.

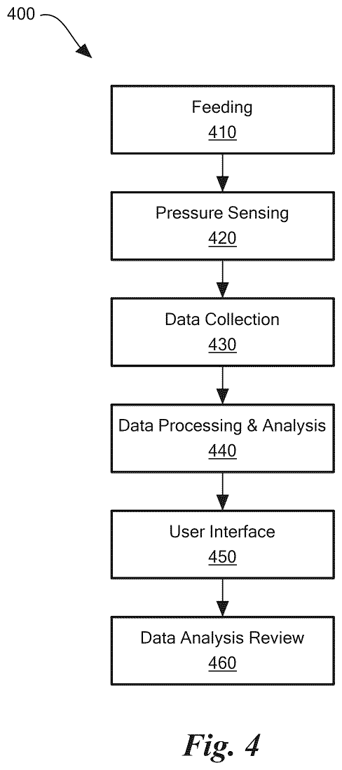

FIG. 4 illustrates an example process 400 for tracking feeding activity in accordance with various illustrative embodiments. The process 400 represents a high-level operational flow for utilizing the system 100 to monitor and analyze feeding dynamics, whether during breastfeeding or bottle feeding.

The process begins at block 410 when the system 100 is actively engaged during a feeding session. In various embodiments, this may involve positioning the housing 110 over a breast or integrating it with a bottle nipple. At this stage, the sensors 120-150 are in contact with the baby's oral structures (e.g., tongue, lips) and begin detecting biomechanical interactions such as pressure, suction, and compression forces.

At block 420, the sensors continuously capture pressure readings at their respective locations. These readings may include:

Dynamic Pressure Profiles: Variations in pressure over time corresponding to suck-swallow-breathe cycles.

Peak and Average Forces: Maximum compression during latch and sustained pressure during feeding.

Temporal Patterns: Frequency and rhythm of tongue and lip movements.

In some embodiments, additional parameters such as suction strength or vacuum pressure may also be measured to provide a comprehensive feeding assessment.

At block 430, the sensed data is transmitted to a data collection device. This device may include:

Signal Conditioning Modules: Filtering noise and normalizing sensor outputs.

Data Storage: Logging time-series data for later review or clinical analysis.

Connectivity Features: Wired transmission via cable 170 or wireless protocols (e.g., Bluetooth®, Wi-Fi, NFC) for real-time monitoring on a mobile application or cloud platform.

At block 440, the collected data undergoes computational analysis. In various embodiments, the processing may include:

Comparative Analysis: Evaluating pressure readings across sensors to determine latch symmetry and effectiveness.

Pattern Recognition: Identifying rhythmic feeding cycles and detecting anomalies such as weak suckling or inconsistent latch.

Scoring Algorithms: Generating a quantitative score based on empirical data, clinical benchmarks, or machine learning models trained on normative feeding patterns.

For example, the algorithm may compute ratios of anterior tongue movement (sensor 130) to posterior tongue movement (sensor 120) and lip seal integrity (sensors 140 and 150) to derive a composite latch score.

At block 450, processed data and computed metrics are displayed on a user interface. This interface may be:

Mobile Application: Providing real-time feedback and historical trends.

Clinical Dashboard: Enabling healthcare professionals to monitor multiple patients.

Device Screen: Offering immediate visual indicators (e.g., color-coded latch quality).

At block 460, caregivers or professionals review the displayed results to determine next steps. Actions may include:

Adjusting feeding technique.

Scheduling a lactation consultation.

Logging data for longitudinal growth and development tracking.

In various embodiments, a final score may be presented on a scale (e.g., 0-100), where:

0 indicates no functional movement or latch.

100 represents optimal latch and feeding efficiency.

Scores may be derived empirically (e.g., clinical trials, published data), formulaically, or through hybrid approaches. For instance, a score of 74 may correspond to one or more of the following:

130% movement score for anterior tongue activity.

70% movement score for posterior tongue activity.

80% lip seal integrity.

Such granular metrics enable targeted interventions and personalized feeding plans. In various embodiments, the difference in muscle mechanics may be compared and contrasted based on different feeding outputs (e.g. bottle feeding and breastfeeding).

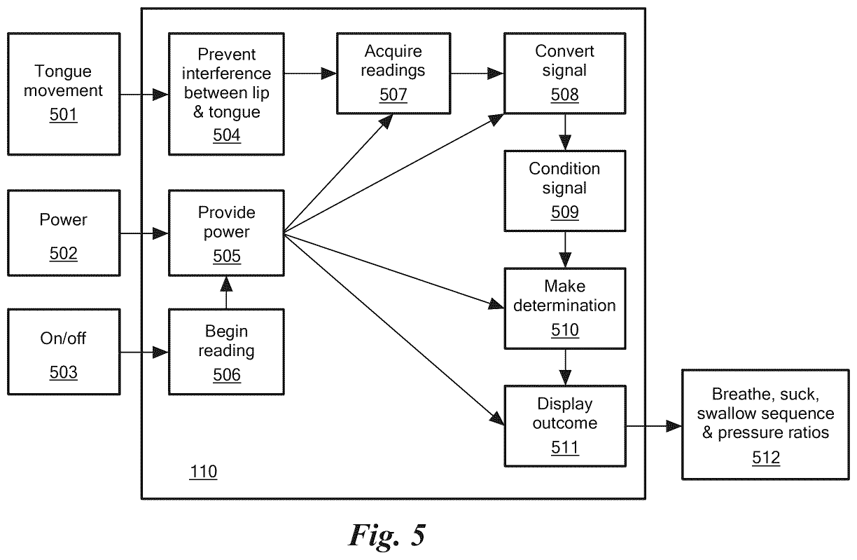

FIG. 5 illustrates an example schematic block diagram of the system 100 of FIG. 1 in accordance with various illustrative embodiments. The diagram represents the functional architecture of the system, showing how sensor data flows through various processing stages to produce actionable outcomes.

The system 100 includes a tongue movement block 501, which may act to prevent interference between the lip and tongue during feeding. This block may implement algorithms or mechanical constraints to ensure accurate detection of tongue motion without signal contamination from lip pressure. In some embodiments, the tongue movement block may perform one or more of the following:

Apply signal isolation techniques to differentiate tongue pressure from lip compression.

Use sensor fusion algorithms to combine readings from sensors 120 and 130 for posterior and anterior tongue regions.

Provide real-time feedback to caregivers if tongue movement patterns indicate improper latch or restricted mobility.

The system includes a power switch 502 that supplies electrical power to the system at block 505. This may involve a battery source, rechargeable module, or wired power input.

An On/Off Switch 503 initiates system operation at block 506, enabling sensors and data acquisition modules. In some embodiments, the switch may include an LED indicator or haptic feedback to confirm activation.

An acquire readings block 507 interfaces with the sensors to collect raw pressure data. This block may perform one or more of the following:

Implement sampling protocols to capture high-resolution data at predefined intervals.

Synchronize readings across multiple sensors to maintain temporal accuracy.

Include error-checking routines to detect sensor malfunctions or signal anomalies.

A convert signal block 508 transforms raw sensor outputs (e.g., analog voltage) into digital signals suitable for processing. This may involve: analog-to-digital conversion (ADC) and/or calibration routines to normalize sensor outputs.

A condition signal block 509 applies filtering and scaling to prepare signals for analysis. In some embodiments, this may include low-pass filtering to remove noise, dynamic range adjustment for consistent interpretation, and/or compensation for environmental factors such as temperature or humidity.

A make determination block 510 processes conditioned signals to compute feeding metrics. In various embodiments, this may include one or more of the following:

Calculating pressure ratios between tongue and lip sensors.

Identifying suck-swallow-breathe sequences.

Generating a composite latch score based on predefined algorithms or machine learning models.

A display outcome block 511 presents results on a user interface. Outcomes may include one or more of the following:

Latch Quality Score (e.g., 0-100 scale).

Functional Indicators such as tongue mobility, lip seal integrity, and suction strength.

Sequence Analysis showing rhythmic patterns of breathing, sucking, and swallowing.

Pressure Ratios (512): Displayed as numerical values or graphical plots for detailed interpretation by caregivers or clinicians.

In various embodiments, the system 100 may support wireless connectivity for transmitting processed data to mobile applications or cloud platforms. Additionally, adaptive algorithms may be employed that learn from historical feeding sessions to improve scoring accuracy. Alerts or recommendations for corrective actions may be provided if latch quality falls below a threshold.

FIG. 6 shows an example user interface 600 with data available to parents/professionals to review data collected in accordance with illustrative embodiments. In section 610, an application name for the application being utilized may be displayed. Section 620 displays a name of a patient (e.g., baby's name) for which data is being collected.

A daily average of scores may be displayed in section 630, and an overall feeding score may be displayed in section 640.

The user interface 600 may be displayed on a phone or any display where a user may desire to view the information, such as a computer screen.

The system senses pressure, enabling it to be used as any of a variety of tools, such as a research tool, a diagnostic tool, a learning tool, etc. The sensors may be embedded near the tongue/lips or mechanically actuated, depending on the sensors implemented. Those skilled in the art may select sensors other than those described and, in fact, may position the noted sensors in different locations. Accordingly, while illustrative embodiments may involve distinct sensors and specific configurations and modalities, those in the art may use other sensors and thus, the sensors described herein are not intended to limit all embodiments. Further data may also be provided to a user in the form of waveforms and plots that may assist in a determination of whether a baby is developing proper feeding habits or may need intervention.

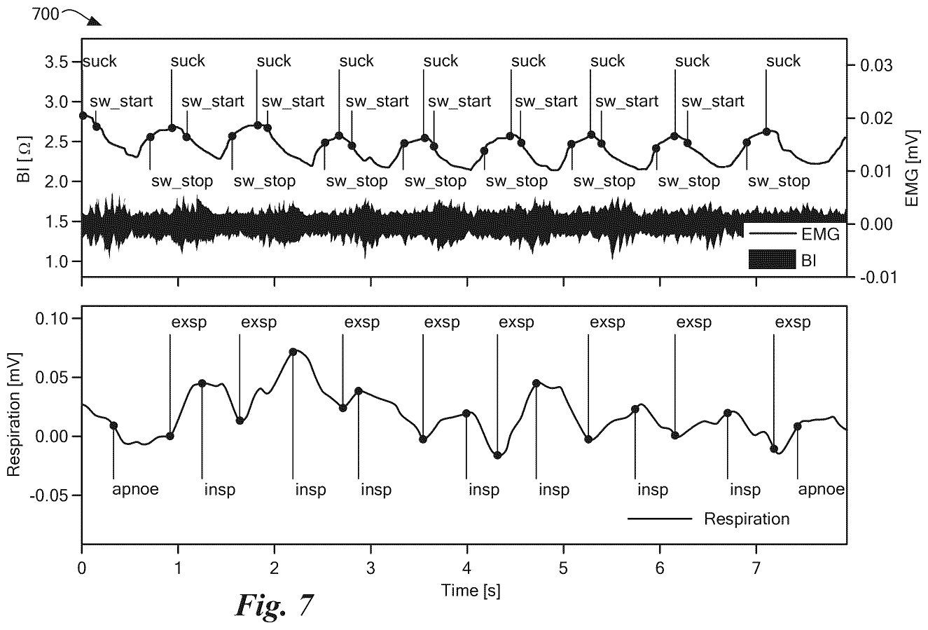

For example, FIG. 7 shows an exemplary waveform 700 with annotations of a swallowing and breathing pattern for a baby while breastfeeding in accordance with illustrative embodiments.

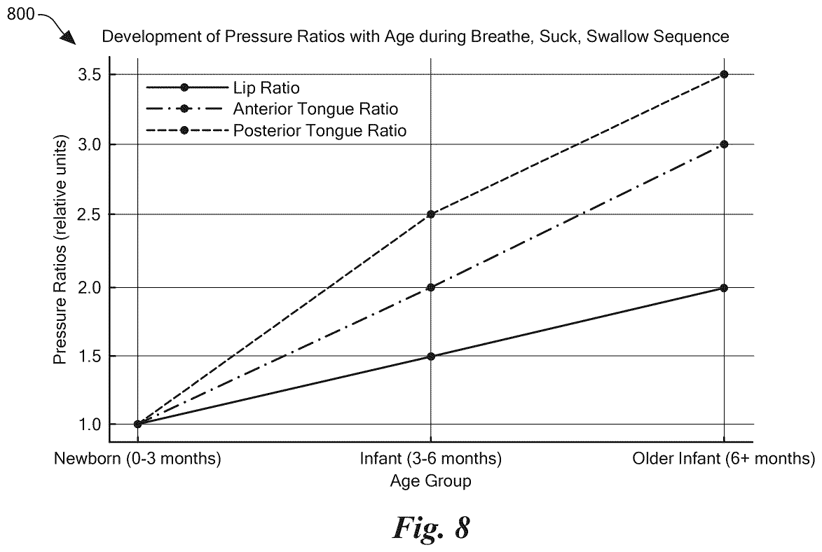

FIG. 8 shows an exemplary plot 800 of ideal ratio of feeding phases over development time in accordance with illustrative embodiments.

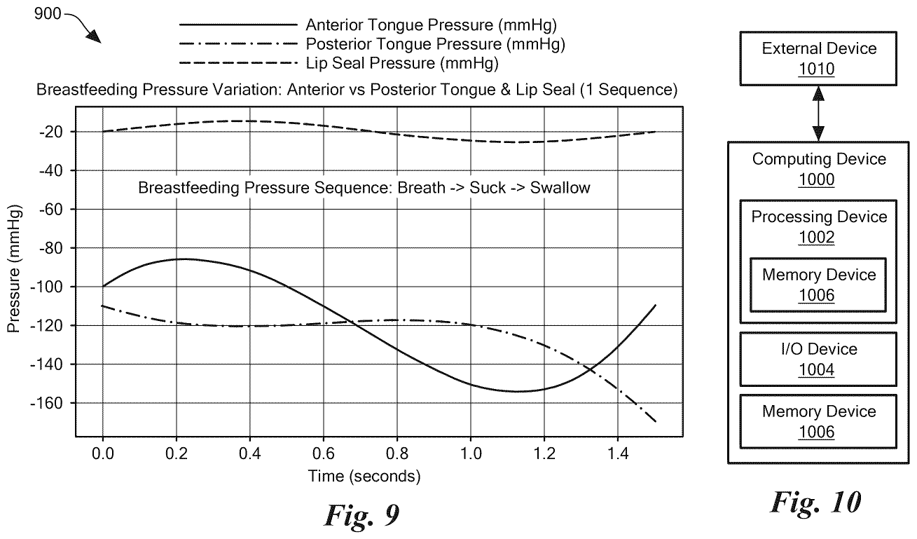

FIG. 9 shows an exemplary plot 900 of expected pressure of lip and tongue during one sequence of feeding in accordance with illustrative embodiments.

Described below is various information relating to anatomy and physiology of feeding. The breathe, suck, swallow sequence is a crucial rhythm that babies develop to feed effectively and safely. It involves coordinating sucking milk, swallowing it, and pausing to breathe-all in a continuous, synchronized cycle.

### Breathe, Suck, Swallow Sequence Basics

Sucking: The baby latches onto the nipple (breast or bottle) and creates suction by moving their tongue and jaw. This draws milk into the mouth.
Swallowing: After the mouth fills with milk, the baby's throat muscles contract to move milk down the esophagus. During swallowing, the soft palate lifts to close off the nasal passage, preventing milk from entering the nasal cavity.
Breathing: The baby pauses momentarily to breathe through their nose, as they cannot breathe while swallowing. After swallowing, they resume the cycle of sucking, swallowing, and breathing.

This sequence requires precise coordination, especially in young infants, and is essential for preventing milk from entering the airway, which could lead to choking.

### Development of Breathe, Suck, Swallow in Newborns

Newborns (0-3 months): Newborns have an instinctual suck reflex but must develop the coordination to manage the full sequence smoothly. Their feeding sessions are often slower, with frequent pauses to catch their breath. Early on, they may exhibit irregular rhythms as they learn to synchronize each action.
   Ratio: In very young infants, the breathe, suck, swallow pattern often follows a 1:1:1 ratio-one suck, one swallow, and one breath. This pattern is slower and more deliberate, as their muscle coordination is still developing.
   Pace: Newborns typically complete around one to two sucks per second, which can vary with feeding method and flow rate. For breastfeeding, milk flow is influenced by let-down, allowing pauses that naturally support breathing.
   Breathing Pauses: Because newborns are still learning to synchronize, they may need to pause more frequently for breaths between cycles, which is why feedings are often longer and include more breaks.
3-6 Months: Around this age, babies become more efficient with the suck-swallow-breathe sequence. The process becomes smoother and faster as they gain better control over the muscles involved. Babies generally begin to feed more consistently and handle larger milk flows, either from a breast or bottle, as they develop this coordination.
   Ratio: By 3 to 6 months, infants may shift to a 2:1 or 3:1 suck-to-swallow ratio. This means they take two to three sucks, followed by a swallow, and then a breath. With more developed coordination, they can handle a slightly faster pace and multiple sucks before each swallow.
   Pace: At this stage, babies tend to suck around two sucks per second and may become more efficient in feeding, especially as their oral muscles grow stronger. Breastfed babies regulate their pace in line with milk flow, while bottle-fed babies may need adjustments in bottle nipple flow to match their developmental abilities.
   Breathing Pauses: Babies may now feed with fewer breathing breaks, completing multiple suck-swallow cycles before pausing to breathe. However, they still take regular pauses, especially at the beginning and end of the feed.

### Older Infants (6+ Months)

Ratio: Older infants typically develop a 3:1 or 4:1 suck-to-swallow ratio, meaning they can handle up to four suckles per swallow with fewer breaths. This efficiency in the sequence reflects their stronger muscles and enhanced coordination.
Pace: At this stage, babies might continue at about two to three sucks per second, but they are more adept at adjusting their rhythm according to the feeding context, whether breastfeeding or bottle feeding.
Breathing Pauses: Older infants generally take fewer pauses during feeding, especially as they approach the ability to drink from a cup or eat soft solids. Their breathing during feeding has also become more regulated, allowing longer sequences before pausing.

In each cycle of the breathe, suck, swallow sequence, these structures work in perfect synchronization. Together, they manage milk flow, guide it toward the esophagus, and allow quick pauses for breathing, ensuring efficient and safe feeding for the baby. See Table 1 below.

TABLE 1
Descriptions of the muscles used during each feeding sequence.

| | | Anterior | Posterior | Pressure |
|---|---|---|---|---|
| Phase | Lips | Tongue | Tongue | on Breast |
| Suck | Forms a tight | Moves | Stabilizes nipple; | Creates a vacuum |
| | seal around the | rhythmically in | directs milk flow | in the mouth, |
| | nipple; outward | a wave-like | toward back of | applying negative |
| | flanging (lip | motion, creating | the mouth | pressure to |
| | turning out) to | suction to draw | | draw milk out |
| | secure latch | milk | | of the breast |
| Swallow | Maintains seal | Pauses suction | Elevates to push | Minimal |
| | but minimal | momentarily as | milk down the | pressure on the |
| | movement | milk flows | throat, coordinating | breast, as |
| | during swallow | toward back | with soft palate | sucking action |
| | | of mouth | closure to prevent | pauses briefly |
| | | | milk from entering | |
| | | | the nasal cavity | |
| Breathe | Slightly relaxes | Rests low and | Rests low in the | No pressure on |
| | to allow a brief | forward in the | mouth, allowing | the breast, as |
| | intake of air | mouth, away | airway to remain | suction is |
| | | from the hard | open for nasal | released to |
| | | palate | breathing | allow breathing |

During breastfeeding, the functional pressure exerted by both the posterior and anterior tongue may not be a single, fixed number, but rather a dynamic range of pressure depending on the sucking action, with the posterior tongue generally generating slightly higher pressure than the anterior tongue, creating a vacuum to effectively extract milk from the breast; typically, the peak negative pressure during a suckle can range from −110 to −170 mmHg.
Pressure variation:
The pressure fluctuates throughout the suckling cycle, with higher pressure during the active sucking phase and lower pressure during the pause between sucks.
Posterior tongue dominance:
The posterior tongue plays a more significant role in generating the necessary pressure to draw milk out, due to its larger surface area contacting the breast.
Anterior tongue function:
The anterior tongue helps to maintain the seal around the nipple, facilitating the negative pressure created by the posterior tongue.

### Lips

Sealing and Stability: The lips form a firm seal around the breast or bottle nipple to prevent milk leakage. This seal allows for efficient suction, as it keeps air from entering the mouth and affecting the pressure needed to draw in milk.
Flanging: During breastfeeding, the upper and lower lips often turn outward, a movement called “flanging.” This outward seal enhances suction by creating a tighter vacuum.
Minimal Movement: The lips remain relatively stable during feeding, maintaining their seal to stabilize the nipple. Any loss of the seal could disrupt the sucking rhythm and introduce air, which can lead to discomfort or gas.

### Anterior (Front) Tongue

Generating Suction: The front of the tongue presses against the nipple and creates a wave-like, peristaltic movement (a ripple-like motion) to draw milk from the breast or bottle. This movement is essential for creating the suction pressure needed to extract milk.
Controlling Milk Flow: As the anterior tongue presses and releases, it regulates the flow of milk, which is particularly important for breastfeeding, where milk flow can vary with let-down. The tongue's movements help control the milk pace, preventing too much milk from entering the mouth at once.
Stabilizing and Positioning the Nipple: The front tongue helps keep the nipple in the correct position within the mouth, creating a stable point of contact. This positioning allows the baby to manage the suck-swallow rhythm efficiently.

### Posterior (Back) Tongue

Propelling Milk for Swallowing: Once milk is drawn into the mouth, the posterior tongue elevates and propels it toward the back of the throat for swallowing. This movement is timed with the closure of the airway to ensure milk flows safely down the esophagus.
Swallow Coordination: The back of the tongue works in tandem with the soft palate, which lifts to close off the nasal passage during swallowing. This coordination prevents milk from entering the airway and ensures that milk is directed into the esophagus.
Pacing and Timing: The posterior tongue's actions are critical for the timing of each swallow. After each swallow, the posterior tongue relaxes briefly, allowing the baby to take a quick breath before initiating the next cycle of suck and swallow.

### Summary of the Lips, Anterior, and Posterior Tongue Functions

Lips: Seal and stabilize the nipple, helping to maintain a closed system that prevents milk leakage and air intake.
Anterior Tongue: Generates suction through rhythmic movements, controls milk flow, and stabilizes the nipple within the mouth.

Posterior Tongue: Elevates and pushes milk toward the esophagus for swallowing, coordinating with the soft palate to ensure safe swallowing without disrupting the breathing cycle.

FIG. 10 shows an example computing device in accordance with various embodiments. For example, FIG. 10 schematically shows a computing device 1000 in accordance with various embodiments. The computing device 1000 is one example of a computing device which is used to perform one or more operations of the process/method illustrated in FIG. 1. The computing device 1000 includes a processing device 1002, an input/output device 1004, and a memory device 1006. The computing device 1000 may be a stand-alone device, an embedded system, or a plurality of devices configured to perform the functions described with respect to one of the components of power network 100. Furthermore, the computing device 1000 may communicate with one or more external devices 1010. In various embodiments, the computing device 1000 may communicate with the system 100 of FIG. 1, which may be the external device 1010. In various embodiments, the computing device 1000 may also reside within the system 100 or external to the system 100.

The input/output device 1004 enables the computing device 1000 to communicate with an external device 1010. For example, the input/output device 1004 may be a network adapter, a network credential, an interface, or a port (e.g., a USB port, serial port, parallel port, an analog port, a digital port, VGA, DVI, HDMI, FireWire, CAT 5, Ethernet, fiber, or any other type of port or interface), among other things. The input/output device 1004 may be comprised of hardware, software, or firmware. The input/output device 1004 may have more than one of these adapters, credentials, interfaces, or ports, such as a first port for receiving data and a second port for transmitting data, among other things.

The external device 1010 may be any type of device that allows data to be input or output from the computing device 1000. For example, the external device 1010 may be a meter, a control system, a sensor, a mobile device, a reader device, equipment, a handheld computer, a diagnostic tool, a controller, a computer, a server, a printer, a display, a visual indicator, a keyboard, a mouse, or a touch screen display, among other things. Furthermore, the external device 1010 may be integrated into the computing device 1000. More than one external device may be in communication with the computing device 1000. In various embodiments, the external device 1010 may be the system 100 of FIG. 1 as mentioned above.

The processing device 1002 may be a programmable type, a dedicated, hardwired state machine, or a combination thereof. The processing device 1002 may further include multiple processors, Arithmetic-Logic Units (ALUs), Central Processing Units (CPUs), Digital Signal Processors (DSPs), or Field-programmable Gate Arrays (FPGA), among other things. For forms of the processing device 1002 with multiple processing units, distributed, pipelined, or parallel processing may be used. The processing device 1002 may be dedicated to performance of just the operations described herein or may be used in one or more additional applications.

The processing device 1002 may be of a programmable variety that executes processes and processes data in accordance with programming instructions (such as software or firmware) stored in the memory device 1006. Alternatively or additionally, programming instructions are at least partially defined by hardwired logic or other hardware. The processing device 1002 may be comprised of one or more components of any type suitable to process the signals received from the input/output device 1004 or elsewhere, and provide desired output signals. Such components may include digital circuitry, analog circuitry, or a combination thereof.

The memory device 1006 in different embodiments may be of one or more types, such as a solid-state variety, electromagnetic variety, optical variety, or a combination of these forms, to name but a few examples. Furthermore, the memory device 1006 may be volatile, nonvolatile, transitory, non-transitory or a combination of these types, and some or all of the memory device 1006 may be of a portable variety, such as a disk, tape, memory stick, or cartridge, to name but a few examples. In addition, the memory device 1006 may store data which is manipulated by the processing device 1002, such as data representative of signals received from or sent to the input/output device 1004 in addition to or in lieu of storing programming instructions, among other things. As shown in FIG. 3 20, the memory device 1006 may be included with the processing device 1002 or coupled to the processing device 1002, but need not be included with both.

The operations and blocks of any method or process described above, such as of FIG. 4 are merely illustrative, and variations are contemplated to be within the scope of the present disclosure. In embodiments, the operations and blocks may include other operations not illustrated. In embodiments, the operations and blocks may not include every operation illustrated. In embodiments, the operations and blocks may be implemented in a different order than that illustrated. Such and other embodiments are contemplated to be within the scope of the present disclosure. Persons of skill in the art will appreciate that, although various example components may be described as performing various functions, operations and blocks, other components may perform those functions, operations and blocks described above.

It is contemplated that the various aspects, features, processes, and operations from the various embodiments may be used in any of the other embodiments unless expressly stated to the contrary. Certain operations illustrated may be implemented by a computer executing a computer program product on a non-transient, computer-readable storage medium, where the computer program product includes instructions causing the computer to execute one or more of the operations, or to issue commands to other devices to execute one or more operations.

While the present disclosure has been illustrated and described in detail in the drawings and foregoing description, the same is to be considered as illustrative and not restrictive in character, it being understood that only certain exemplary embodiments have been shown and described, and that all changes and modifications that come within the spirit of the present disclosure are desired to be protected. It should be understood that while the use of words such as “preferable,” “preferably,” “preferred” or “more preferred” utilized in the description above indicate that the feature so described may be more desirable, it nonetheless may not be necessary, and embodiments lacking the same may be contemplated as within the scope of the present disclosure, the scope being defined by the claims that follow. In reading the claims, it is intended that when words such as “a,” “an,” “at least one,” or “at least one portion” are used there is no intention to limit the claim to only one item unless specifically stated to the contrary in the claim. The term “of” may connote an association with, or a connection to, another item, as well as a belonging to, or a connection with, the other item as informed by the context in which it is used. The terms “coupled to,” “coupled with” and the like include indirect connection and coupling, and further include but do not require a direct coupling or connection unless expressly indicated to the contrary. When the language “at least a portion” or “a portion” is used, the item can include a portion or the entire item unless specifically stated to the contrary. Unless stated explicitly to the contrary, the terms “or” and “and/or” in a list of two or more list items may connote an individual list item, or a combination of list items. Unless stated explicitly to the contrary, the transitional term “having” is open-ended terminology, bearing the same meaning as the transitional term “comprising.”

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented as a pre-configured, stand-alone hardware element and/or as preprogrammed hardware elements (e.g., application specific integrated circuits, FPGAs, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

The embodiments of the invention described above are intended to be merely exemplary; numerous variations and modifications will be apparent to those skilled in the art. Such variations and modifications are intended to be within the scope of the present invention as defined by any of the appended claims. It shall nevertheless be understood that no limitation of the scope of the present disclosure is hereby created, and that the present disclosure includes and protects such alterations, modifications, and further applications of the exemplary embodiments as would occur to one skilled in the art with the benefit of the present disclosure

What is claimed is:

1. A system for tracking feeding activity of a baby, comprising:
a housing configured to be positioned over an object for feeding the baby;
a plurality of sensors disposed on or within the housing, the sensors configured to detect pressure exerted by one or more oral structures of the baby during feeding; and
a data collection device operatively coupled to the plurality of sensors, the data collection device configured to receive sensor signals and process the signals to determine one or more feeding metrics,
wherein the data collection device is configured to output the one or more feeding metrics for display to a user, wherein the feeding metrics include one or more of: latch quality, tongue movement, lip seal integrity, or suck-swallow-breathe coordination.

2. The system of claim 1, wherein the housing is configured to be placed over a human breast.

3. The system of claim 1, wherein the housing is configured to be incorporated into a nipple of a baby feeding bottle.

4. The system of claim 1, wherein the housing comprises a first portion proximate to an opening for fluid communication, a second portion radially outward from the first portion, and a third portion distal from the opening, the plurality of sensors being distributed across the first and second portions.

5. The system of claim 1, wherein the plurality of sensors comprises:
a first sensor positioned to detect pressure from a posterior tongue region;
a second sensor positioned to detect pressure from an anterior tongue region;
a third sensor positioned to detect pressure from a lower lip region; and
a fourth sensor positioned to detect pressure from an upper lip region.

6. The system of claim 1, wherein the sensors comprise at least one of:
a fluid-filled sensor configured to convert pressure-induced deformation into an electrical signal; or
a microelectromechanical system (MEMS) sensor embedded within a flexible housing material.

7. The system of claim 6, further comprising a pressure measuring sensor system that measures change in muscle pressure, wherein the pressure measuring sensor system measures one or more of the following: resistance, capacitance, or inductance changes in the baby's orofacial muscle mechanics.

8. The system of claim 1, further comprising one or more holes adapted to facilitate biochemical feedback.

9. The system of claim 1, wherein the data collection device includes signal conditioning circuitry configured to filter noise, normalize sensor outputs, and compute pressure ratios between tongue and lip regions.

10. The system of claim 1, wherein the data collection device is configured to generate a composite feeding score based on empirical data, clinical benchmarks, or machine learning algorithms.

11. The system of claim 1, further comprising a display configured to present real-time feedback including graphical plots of pressure profiles and rhythmic feeding patterns.

12. The system of claim 1, further comprising a tongue movement isolation module configured to prevent interference between tongue and lip pressure signals during feeding analysis.

13. A method for assessing feeding activity of a baby, the method comprising:
positioning a housing over a feeding interface configured to contact one or more oral structures of the baby;
detecting, via a plurality of sensors disposed on or within the housing, pressures exerted by the baby during feeding;
transmitting sensor signals from the plurality of sensors to a data collection module;
processing the sensor signals to generate one or more feeding metrics based on detected pressure patterns; and
outputting the one or more feeding metrics for presentation to a user, the feeding metrics comprising at least one of latch quality, tongue movement, lip seal integrity, or suck-swallow-breathe coordination.

14. The method of claim 13, wherein positioning the housing comprises positioning the housing over a human breast.

15. The method of claim 13, wherein positioning the housing comprises integrating the housing into a nipple of a baby feeding bottle.

16. The method of claim 13, wherein detecting pressures comprises detecting pressures across a proximal region near a fluid opening and an intermediate region radially outward from the proximal region.

17. The method of claim 13, wherein detecting pressures comprises:
detecting pressure applied by a posterior region of the baby's tongue;
detecting pressure applied by an anterior region of the baby's tongue;
detecting pressure applied by the baby's lower lip; and
detecting pressure applied by the baby's upper lip.

18. The method of claim 13, wherein detecting pressures comprises receiving signals from at least one of:
a fluid-filled pressure sensor configured to convert deformation into an electrical response; or
a microelectromechanical system (MEMS) based pressure sensor embedded in a flexible portion of the housing.

19. The method of claim 13, further comprising conditioning the sensor signals by filtering noise, normalizing amplitudes, and calculating pressure ratios between tongue-associated and lip-associated sensors.

20. The method of claim 13, wherein processing the sensor signals comprises generating a composite feeding score using empirical training sets, clinical reference values, or a machine-learning model.

21. The method of claim 13, further comprising displaying real-time graphical representations of pressure profiles, rhythmic feeding cycles, or time-based feeding trends.

22. A computer program product for use on a computer system, the computer program product comprising a tangible, non-transient computer usable medium having computer readable program code thereon, the computer readable program code comprising:
program code for positioning a housing over a feeding interface configured to contact one or more oral structures of the baby;
program code for detecting, via a plurality of sensors disposed on or within the housing, pressures exerted by the baby during feeding;
program code for transmitting sensor signals from the plurality of sensors to a data collection module;
program code for processing the sensor signals to generate one or more feeding metrics based on detected pressure patterns; and
program code for outputting the one or more feeding metrics for presentation to a user, the feeding metrics comprising at least one of latch quality, tongue movement, lip seal integrity, or suck-swallow-breathe coordination.
